(19) United States
(12) Patent Application Publication
(10) Pub. No.: US 2011/0301687 A1
(43) Pub. Date: Dec. 8, 2011

(54) DEVICE SUITABLE FOR IMPLANTATION AT A DESIRED POSITION IN A LUMEN OF WARM-BLOODED LIVING BEING, AS WELL AS A HOLDER SUITABLE FOR SUCH A DEVICE

(75) Inventor: Ernst Stefan Christiaan Van Waalwijk Van Doorn, Delft (NL)

(73) Assignee: APPLIED MEDICAL DEVELOPMENTS BV, Delft (NL)

(21) Appl. No.: 13/143,321

(22) PCT Filed: Dec. 28, 2009
(86) PCT No.: PCT/NL09/00272

§ 371 Date: Aug. 5, 2011

(30) Foreign Application Priority Data
Jan. 7, 2009 (NL) 1036388

(51) Int. Cl. A61F 2/82

(52) U.S. Cl. 623/1.11

(57) ABSTRACT

A device suitable for implantation at a desired position in a lumen of a warm-blooded living being, which device comprises a tubular holder to be fixed in the lumen and at least one function element to be detachably fitted in the holder. The holder comprises a deformable tube which, in a first form thereof, is provided with slits, which holder can be made to expand in use from the first form to a second, expanded form once it has been implanted at the desired position in the lumen, with the slits forming mesh-shaped openings. The holder, in the expanded form thereof, further comprises a first portion having a reduced first diameter, in which the function element can be detachably fitted.

The invention relates to a device suitable for implantation at a desired position in a lumen of a warm-blooded living being, which device comprises a tubular holder to be fixed in the lumen and at least one function element to be detachably fitted in the holder.

The invention also relates to a holder suitable for such a device.

In such a device, which is known from U.S. Pat. No. 4,679,546, the tubular holder comprises a flange extending transversely to the central axis of the holder. The holder is surgically implanted into the human body near the junction between the urinary bladder and the urethra, where the flange abuts against the bladder wall, whilst the tubular holder extends into the urethra. Subsequently, a shut-off valve is fitted in the holder as a function element, which valve shuts off the urethra. The user can open the valve by exerting pressure on the urinary bladder, as a result of which the urinary bladder is emptied via the open valve and the urethra. Once the flow of urine and the pressure are sufficiently low, the valve is closed again.

Although the device described in the aforesaid US patent in itself functions well, the placement of the holder near the junction between the urinary bladder and the urethra is a relatively complex operation.

In addition, the device is not suitable for placement in other lumina of a human or an animal, for example in veins, vessels, the trachea, the oesophagus, etc.

The object of the invention is to provide a device in which the holder can be placed in a suitable lumen in a relatively simple manner, after which a function element can be fitted in the holder.

This object is accomplished with the device according to the invention in that the holder comprises a deformable tube which, in a first form thereof, is provided with slits, which holder can be made to expand in use from the first form to a second, expanded form once it has been implanted at the desired position in the lumen, with the slits forming mesh-shaped openings, which holder, in the expanded form thereof, further comprises a first portion having a reduced first diameter, in which the function element can be detachably fitted.

Since the holder is expandable, the holder can be displaced through the lumen to the desired position in the lumen in the collapsed first form thereof, whereupon the holder can be made to expand to its expanded form at the desired position.

The expandable holder may be made of a material having elastic and/or plastic properties, for example.

As a result of the presence of the mesh-shaped openings formed in the holder, the lumen tissue will easily grow into the holder, so that a strong fixation of the holder in the lumen is realised. On the one hand this is important in order to prevent spontaneous migration of the holder in the lumen. On the other hand a proper fixation of the holder in the lumen is important in order to be able to fit the function element in the holder and remove it from the holder without the holder being displaced as well.

Since the function element is detachably connected to the reduced-diameter first portion, the function element can be fitted in the holder in a simple manner. In addition, the urinary bladder remains accessible via the passage in the holder for diagnostic and interventional purposes.

The function element may be connected to the holder by means of a detachable clamped connection, for example.

Removal of the holder is possible in that it is provided with weakened spots, so that the holder will break into pieces upon being forcedly deformed, which pieces are removed with a removing tool. It is also possible to cut the holder into pieces by means of a laser device.

One embodiment of the device according to the invention is characterised in that the holder, in the expanded form thereof, comprises a central cylindrical portion which is connected on a first side thereof to the first portion having a reduced first diameter, which is connected to a funnelled portion on a side remote from said cylindrical portion, which cylindrical portion is connected on a second side remote from said first side to a second portion having a reduced second diameter, which is connected to a funnelled portion on a side remote from said cylindrical portion. Preferably, said second diameter being larger than said first diameter.

The function element to be fitted in the holder can be positioned in the first portion having a reduced first diameter via the second portion having a reduced second diameter in the cylindrical portion.

As a result of the provision of the central cylindrical portion, which has a diameter larger than the first and the second diameter, a firm clamping engagement in the lumen is effected and the ingrowth of tissue is stimulated, whilst sufficient space remains for the function element. This shape of the holder prevents migration of the holder towards the bladder as a result of any atrophy of the urinary bladder neck tissue.

Yet another embodiment of the device according to the invention is characterised in that the holder is tubular in shape in the first form thereof, whilst at least one end is funnel-shaped in the expanded form.

The holder, which is tubular in shape in the collapsed form thereof, can be displaced through the lumen to the desired position therein in a relatively simple manner. The funnel-shaped end (in the expanded form) will keep the holder clamped in position in the lumen.

The invention will now be explained in more detail with reference to drawings, in which:

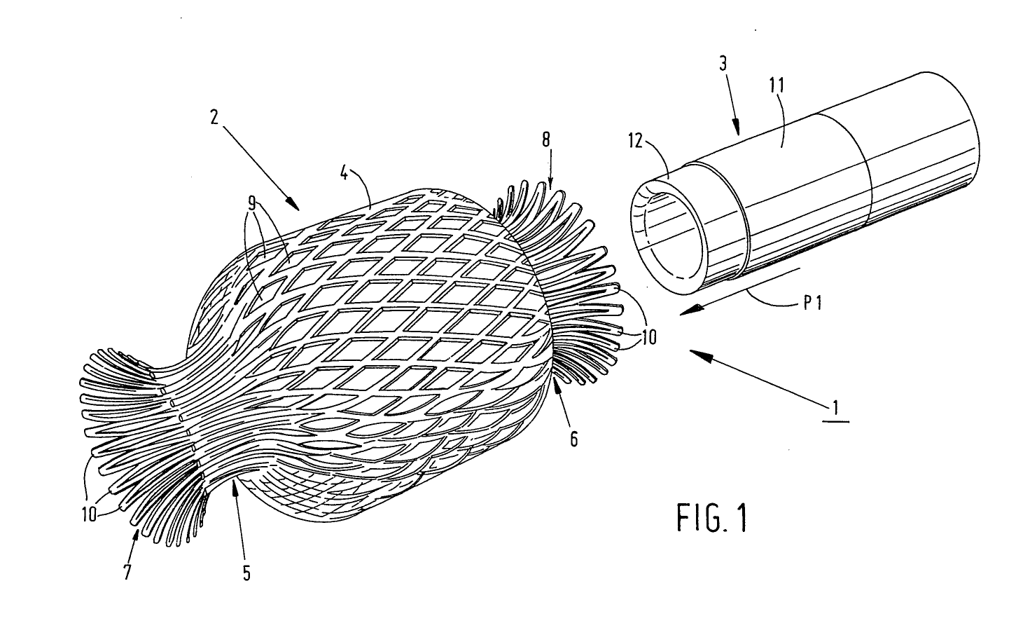

FIG. 1 is a perspective view of a holder and a function element of a device according to the invention, in which the function element is not positioned in the holder yet;

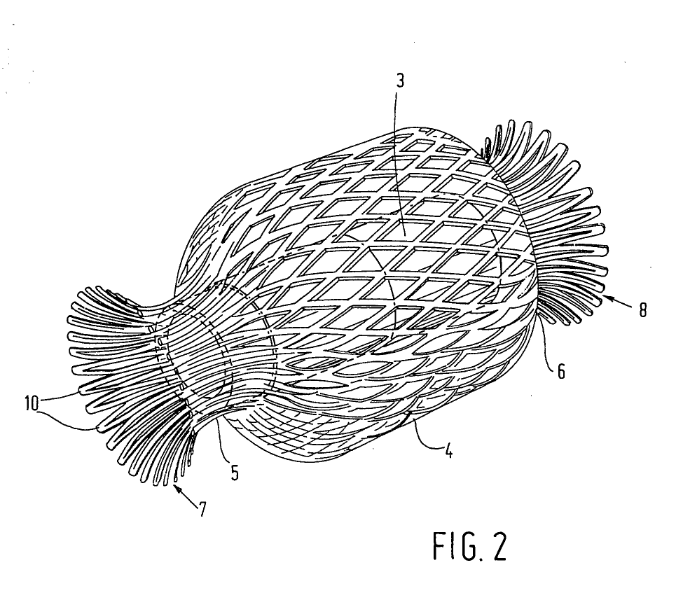

FIG. 2 is a view of the holder and the function element of FIG. 1, in which the function element is positioned in the holder;

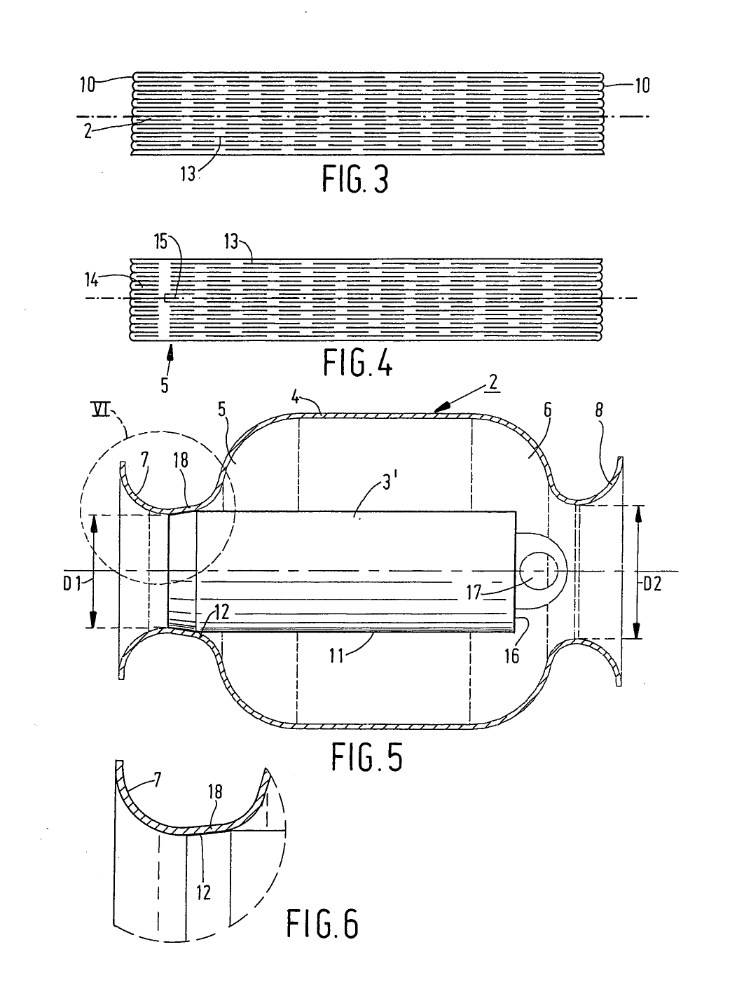

FIG. 3 shows the holder of FIG. 1 in the first, non-expanded form thereof;

FIG. 4 shows another embodiment of a holder according to the invention in the first, non-expanded form thereof;

FIG. 5 is a cross-sectional view of a device according to the invention;

FIG. 6 shows a detail VI of the device shown in FIG. 5;

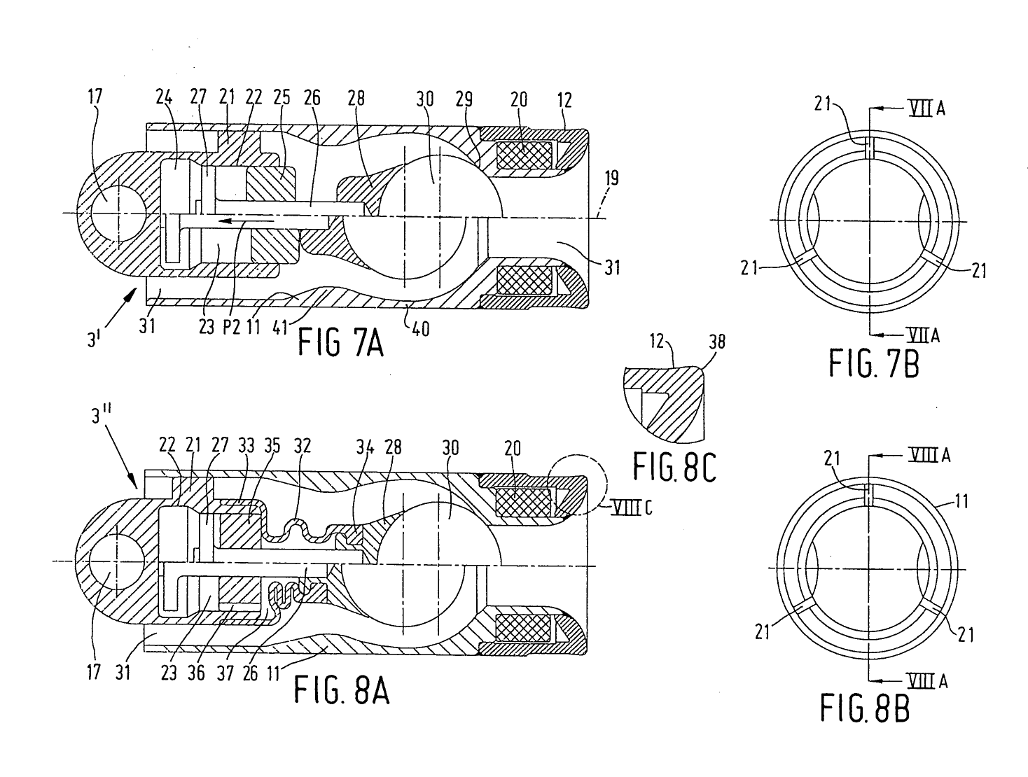

FIG. 7A is a cross-sectional view, partially in a closed position and partially in an open position, of a function element comprising a valve system;

FIG. 7B is a side view of the function element shown in FIG. 7A;

FIG. 8A is a cross-sectional view, partially in a closed position and partially in an open position, of a function element comprising another embodiment of a valve system;

FIG. 8B is a side view of the function element shown in FIG. 8A;

FIG. 8C shows a detail VIII C of the function element shown in FIG. 8A;

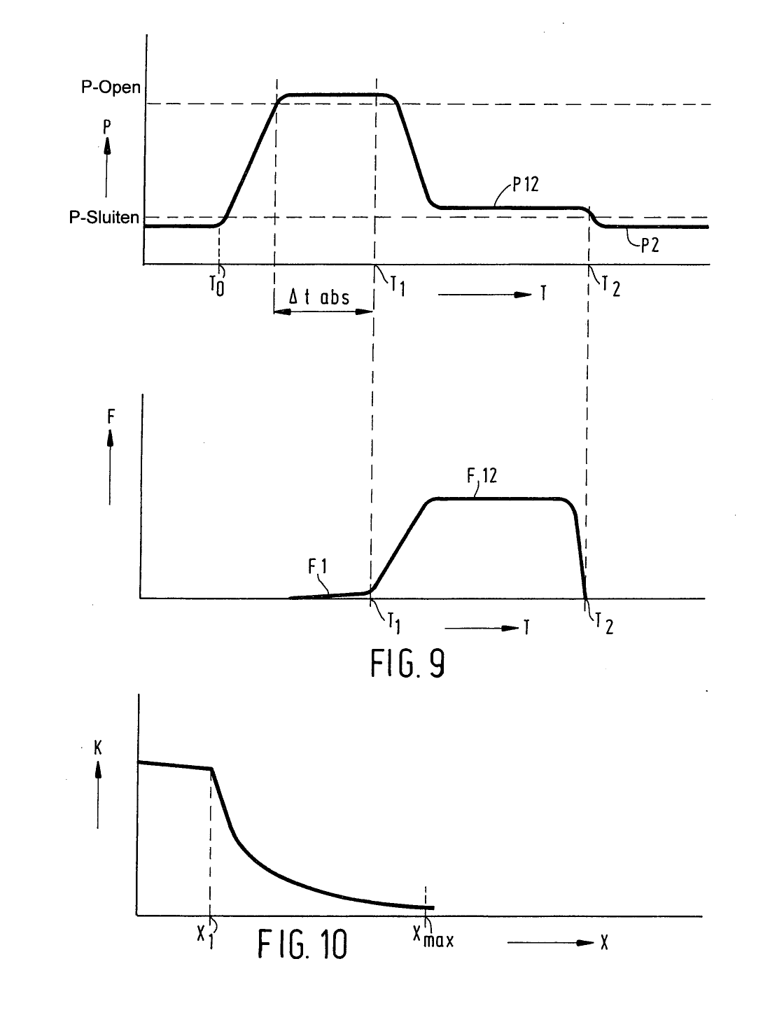

FIG. 9 shows graphs in which the pressure on and the liquid flow through the function element shown in FIG. 7A are plotted against time;

FIG. 10 shows a force-position graph of the damper of the function element shown in FIG. 7A.

Like parts are indicated by the same numerals in the figures.

FIGS. 1-3 are views of a device 1 according to the invention, which comprises a holder 2 and a function element 3 to be fitted in the holder 2.

The holder 2 is made of a tube of a shape-memorizing material, such as a TiNi alloy. The tube is circumferentially provided with a slot pattern. In the first form, said slot pattern comprises slits 13, which form the openings 9 in the expanded form. The holder 2 can expand from the collapsed, first form of the holder 2 that is shown in FIG. 3 to the expanded, second form of the holder 2 that is shown in FIG. 1 under the influence of a temperature increase.

In the expanded form, the holder 2 comprises a central, cylindrical portion 4, which is reduced in diameter at both ends to form first and second reduced-diameter portions 5, 6. Each reduced-diameter portion 5, 6 is connected to a funnelled portion 7, 8 on a side remote from the cylindrical portion 4.

The tube has expanded relatively much in the cylindrical part 4, so that relatively large openings 9 have formed, through which tissue can easily grow in.

Projections 10 extending in a direction away from the central axis through the slot pattern material are formed near the funnel-shaped portions 7, 8, which projections can press themselves into the lumen tissue like hook-like claws 10.

The function element 3 shown in FIGS. 1 and 2 comprises a cylindrical housing 11, which comprises a reduced-diameter portion near a first end 12, which reduced-diameter portion can be placed in the first reduced-diameter portion 5 with a clamping fit by moving the function element 3 through the second reduced-diameter portion in the direction indicated by the arrow P1.

FIG. 2 is a perspective view of the holder 2 with the function element 3 fitted therein.

FIG. 3 shows the holder 2 in the first, non-expanded form, in which the holder 2 has the shape of a cylindrical sleeve, with slits 13 being provided in the wall of the cylindrical sleeve, which slits form the slot pattern. After expansion of the holder 2 from the form shown in FIG. 3 to the expanded form shown in FIG. 1, said slits 13 form the openings 9.

As long as the holder 2 is stored at a relatively low temperature, the holder 2 will retain the tubular form shown in FIG. 3.

Said tubular form is also retained by using a tubular insertion tool, in which the holder is temporarily confined. In said tubular form, the holder 2 can be transported through channel-shaped passages in the body and through the desired lumen to the desired position in the lumen by means of said tubular insertion tool. After removal of the insertion tool, the holder 2 will be heated to about 37° C. by the body temperature, causing the shape-memorising metal to deform and assume the shape of the holder 2 that corresponds to said temperature, which is shown in FIG. 1. Upon said deformation, the cylindrical portion 4 is pressed against the inner wall of the lumen, causing the projections 10 to be pressed into the lumen wall. The projections 10 directly cause the holder 2 to be anchored in the lumen. After implantation, the lumen tissue can start to grow through the openings 9, thus further anchoring and sealing the holder 2 in the lumen.

FIG. 4 shows another embodiment of a holder 14 according to the invention, which is provided with a pair of tongues 15 near the first portion 5 to be formed. In the expanded form, the holder 14 has a shape which substantially corresponds to the shape of the holder 2 that is shown in FIG. 1. After deformation of the holder 14 to the expanded form, the tongues 15 will face towards the central axis of the holder 14.

In the expanded form, the tongues 15 are intended for attaching or securing a function element corresponding to the function element 3 thereto. The function element is provided with means that mate with the tongues 15.

FIG. 5 is a cross-sectional view of the holder 2 shown in FIG. 1, with a function element 3′ fitted therein. The function element 3′ is different from the function element 3 in that it is provided with an eye 17 at an end 16 remote from the first end 12. The first reduced-diameter portion 5 has a diameter D1 which is smaller than the diameter D2 of the second reduced-diameter portion 6.

As is shown in FIG. 5 and on a larger scale in FIG. 6, the first reduced-diameter portion 5 is provided with a tapered portion 18 opposite the first end 12 of the function element 3′, in which tapered portion the first end 12 of the function element 3′ is accommodated with a clamping fit.

FIG. 7A is a cross-sectional view of the function element 3′, which is provided with a valve system. In this cross-sectional view, the valve system is shown in the closed position above the central axis 19, whilst the valve system is shown in the open position below the central axis 19.

The valve element 3′ comprises the cylindrical valve housing 11, which is provided with the reduced-diameter portion near the first end 12. The cylindrical valve housing 11 is further provided with an annular permanent magnet 20 near the first end 12.

At an end remote from the first end 12, a damping mechanism is mounted in the cylindrical valve housing 11 by means of three radially extending flanges 21. The damping mechanism comprises a cylinder 22, which is provided with the eye 17 on a side remote from the first end 12. The cylinder 22 is further provided with a cylindrical chamber 23, which opens into a larger-diameter cylindrical chamber 24 at an end remote from the first end 12. On a side facing the first end 12, the cylinder 22 is closed by a closing sleeve 25 which is slidably accommodated in the cylinder. A piston rod 26 extends through the sleeve 25, which piston rod is provided with a piston 27 on a side located in the cylinder 22 and with a ball anvil 28 on a side facing the first end 12.

The valve housing 11 is provided with a valve seat 29 near the permanent magnet 20. Disposed between the ball anvil 28 and the valve seat 29 is a metal ball 30.

The operation of the valve element 3′ will now be briefly explained.

The starting point on which the explanation is based is a situation in which the holder 2 is present in a urethra and the valve element 3′ is fitted in the holder 2. In this situation, the first end 12 is positioned closer to the urinary bladder connected to the urethra than the eye 17.

In the closed position of the valve element 3′, the ball 30 abuts against the valve seat 29 and is retained in this position by means of the magnetic force exerted on the ball 30 by the permanent magnet 20. The permanent magnet 20 and the ball also exert an attractive force on the metal ball anvil 28 and/or the piston (rod) 26, 27.

When the person in whom the holder 2 and the valve element 3′ are implanted issues to empty his/her bladder, said person will apply a pressure on his/her bladder by using muscles and/or by applying an external pressure on the urinary bladder.

When a sufficiently large force is exerted on the ball 30 from the urinary bladder, the ball 30 will be pressed in the direction indicated by the arrow P2 against the magnetic force exerted by the permanent magnet 20. As a result, the ball 30 will exert a force on the ball anvil 28, as a result of which force said ball anvil will also be moved in the direction indicated by the arrow P2. As a result of said movement, the piston 27, which is connected to the ball anvil 28 via the piston rod 26, will also move within the cylinder 22. The liquid present in the cylinder chambers 23, 24 however, resist said movement of the piston 27. As a result of the movement of the piston 27, a larger part of the piston rod 26 will be positioned within the cylinder 22. The spacing required for this is obtained in that the sleeve 25 moves in the opposite direction of the arrow P2. Since the cylinder 22 and the piston 27 moving therein retard the movement in the direction indicated by the arrow P2 of the ball anvil 28 and consequently of the ball 30 abutting thereagainst, said cylinder 22 and said piston 27 function as dampers. If the force exerted on the ball 30 from the urinary bladder is sufficiently large and is maintained for a predetermined period of time, the piston 27, the ball anvil 28 connected thereto and the ball 30 will gradually be moved from the closed position to the open position. Once the ball 30 has been moved away from the valve seat 29, urine can flow out through the urethra from the urinary bladder via the now released opening 31.

When the urine flow and the pressure have sufficiently decreased, the permanent magnet 20 will draw the ball 30 against the valve seat 29. The permanent magnet 20 will also exert a magnetic force on the ball anvil 28, as a result of which the ball anvil 28 with the piston rod 26 and the piston 27 connected thereto will start to move in the direction of the valve seat 29. This movement is retarded as a result of the damping effect of the movement of the piston 27 in the cylinder 22, however.

The damping effect that occurs upon movement of the ball 30 from the closed position to the open position has the advantage that when the ball 30 is subjected to short impulses (having a pulse time of at most about 2 seconds), for example when the user coughs or makes a jumping movement, the valve will hardly be opened, if at all, so that undesirable noticeable urine discharge is prevented.

FIGS. 8A-8C show another embodiment of a valve element 3″ according to the invention, which is different from the valve element 3′ shown in FIG. 7A in that the cylinder 22 and the ball anvil 28 are connected by means of a flexible bellows 32, which surrounds the piston rod 26. The flexible bellows 32 is connected to the cylinder with a first end 33 and to the ball anvil 28 with a second end 34. The cylinder 22 is further partially closed on a side remote from the first end 12 by means of a cap 35 that is fixedly connected thereto. Said cap 35 is provided with passages 36, which extend from the cylinder chamber 23 located within the cylinder 22 to the space 37 located between the bellows 32 and the piston rod 26.

When the ball 30 is moved from the closed position to the open position, and vice versa, liquid present in the cylinder chambers 23, 24 and the space 37 is moved back and forth through the passages 36. Said movement has a damping effect on the movement of the piston 27, and consequently on the movement of the ball 30.

FIG. 8C shows the first end 12 of the valve housing 11, which comprises a reduced-diameter portion, whose edge 38 has a slightly larger diameter. Upon insertion of the first end 12 into the portion 18 of the holder 2, a slight decrease of the insertion force is perceived once the edge 38 has moved past the portion 18, which is a sign that the valve element 3′ is correctly positioned in the holder 2.

The flanges 22 are adapted to mate with an insertion tool provided with bayonet-shaped hooks that can be hooked round the flanges 21.

Such a tool can also be used for removing the function element 3′, 3″ from the holder 2.

If said specific tool and is not available when said removal is to take place, it is also possible to engage a hook in the eye 17 and then remove the function element 3, 3′ or 3″ from the holder 2.

FIG. 9 shows the pressure P on and the flow F through the function element 3′ plotted against the time T. At a pressure lower than a pressure P-close, the valve element 3′ is in its closed position. From point in time T0, a user starts to increase the pressure on the valve element 3′ to a pressure higher than a pressure P-open. Said pressure must be maintained for a period of time Δ T-abs so as to move the piston 27 through the chamber 23 of the cylinder 22. Once the urine flow has started, the user may decrease the pressure to, for example, pressure P12, providing that a good liquid flow is maintained. As long as the pressure P12 is maintained, the valve element 3′ will remain in its open position. At moment T2 the flow through the valve element 3′ decreases and/or the pressure P2 exerted on the valve element 3′ decreases to a level below P-close, as a result of which the permanent magnet 20 will automatically move the ball 30 to the closed position. Following that, also the ball anvil 28 will be drawn in the opposite direction of the arrow P2 with a certain delay.

During the period Δ T-abs, the ball 30 is spaced only a short distance from the valve seat 29, so that only a limited flow F1 will flow through the element 3′. A liquid flow F12 through the passage 31 is obtained once the passage 31 is fully open. At point in time T2, when the urine flow and the pressure are sufficiently low, the ball 30 will be automatically drawn against the valve seat 29, as a result of which the flow through the passage 31 will be ended abruptly.

FIG. 10 shows the force K that is required for effecting a particular movement X of the piston 27. As FIG. 10 shows, relatively much force is needed for effecting a movement X1. During said movement, the piston 27 is moved in the chamber 23. Upon further movement of the piston 27, liquid can flow from the chamber 24 around the piston 27 into the chamber 23 with less damping resistance. Upon further movement of the piston 27, the space available around the piston 27 becomes larger and larger, as a result of which the force K required for moving the piston 27 further will decrease until the piston has been moved a maximum distance Xmax.

The graphs shown in FIGS. 9 and 10 only show examples of a possible trend of the force, the flow and the movement.

It is also possible to place the device according to the invention in another passageway in the human body, for example an oesophagus, a vein, a trachea, etc, or in passageways in other living beings.

It is also possible to use a non-return valve instead of a shut-off valve as the element to be detachably fitted in the holder, which valve is implanted in a blood vessel, for example, or in a channel that opens into the stomach. If the device is implanted in a trachea, the element may comprise a respiratory assistance pump. When placed in a vein, the element may be configured as a cardiac assistance pump.

It is also possible to configure the element as a monitoring system, for example for the insulin level in the bloodstream.

Instead of making the holder expandable by using shape-memorizing metals it is also possible to effect expansion of the holder from a collapsed condition upon implantation to an expanded condition after implantation by designing it to have elastic properties. It is also possible to make use of plastic properties of a holder, in which case the holder is inflated to a desired final form at the desired position, using a balloon catheter and an inflation fluid, for example, which expanded form is retained after removal of the catheter and the inflation fluid.

It is also possible to use a combination of elastic and/or plastic properties and shape-memorizing metals.

The shape-memorising metal or material can reach the expanded position as a result of a temperature increase. It is also possible to use a shape-memorizing material, in which case the holder is completely or partially deformed from the expanded position to the collapsed position as a result of a temperature decrease.

It is also possible to fix the holder in position in the lumen by means of a biocompatible glue.

It is also possible to provide the holder with a biocompatible coating.

It is also possible to put a biocompatible viscous liquid, such as a silicone oil, on the holder and/or the element so as to protect the surface against urine and seal the openings 9 near the first reduced-diameter portion 5 during the ingrowth of tissue. This makes it possible to fit the valve element 3′ in the holder 2 directly after placement of the holder 2 in the lumen.

It is possible to provide the function element entirely or partially with a coating that prevents the growth of urine components thereon.

The force with which the ball is drawn against the valve seat can be adjusted in dependence on the user's requirements, for example by adapting the size and the strength of the permanent magnet 20, adapting the spacing between the valve seat 29, adapting the material composition of the ball 30, etc.

If desired, the valve element 3′ may be fixed in the holder 2 in such a manner that the valve element 3′ is pressed out of the holder 2 in case of an undesirably high-pressure in the urinary bladder.

It is possible to design the internal shape of the valve housing 11 and of the permanent magnet such that when pressures are exerted from the urinary bladder when said urinary bladder is being emptied, the ball will undergo a slightly vibrating movement caused by the urine flowing past said ball, as a result of which any growth on the valve elements is removed.

The valve housing 11 may to that end comprise a concave portion 40 and a convex portion 41 opposite the ball 30. The convex portion is located on a side of the concave portion 40 remote from the valve seat 29 (see FIG. 7A). The flow pattern and the liquid pressure through and over the valve element 3′ can be influenced by adapting the curvatures of the concave and convex portions 40, 41.

It is also possible to provide the end of the holder with outwardly deformed protrusions having a shape other than a funnel shape, such that a firm connection with the lumen can be realised.

Various end forms can be realised by varying or partially interrupting the slot pattern in the holder. A suitable alloy selection and treatment furthermore make it possible to realise a holder which can actively assume various end forms, if desired.

Since the function element can be removed from the holder, the function element can be simply renewed every year, for example, or be temporarily removed for maintenance. Moreover, the inner side of the implanted holder 2 can be inspected after removal of the function element and be cleaned and/or worked using specific tools, if desired.

1. A device suitable for implantation at a desired position in a lumen of a warm-blooded living being, comprising a tubular holder to be fixed in the lumen and at least one function element to be detachably fitted in the holder, wherein the holder comprises a deformable tube which, in a first form thereof, is provided with slits, which holder can be made to expand in use from the first form to a second, expanded form when the holder has been implanted at the desired position in the lumen, with the slits forming mesh-shaped openings, which holder, in the expanded form thereof, further comprises a first portion having a reduced first diameter, in which the function element can be detachably fitted.

2. A device according to claim 1, wherein the function element and the holder are fitted together by a clamped connection.

3. A device according to claim 1, wherein the holder is made of a shape-memorizing material, with the holder being expandable to the second, expanded form at body temperature.

4. A device according to claim 1, wherein the holder, in the expanded form thereof, comprises a central cylindrical portion which is connected on a first side thereof to the first portion having a reduced first diameter, which is connected to a first funnelled portion on a side remote from said cylindrical portion, which cylindrical portion is connected on a second side remote from said first side to a second portion having a reduced second diameter, which is connected to a second funnelled portion on a side remote from said cylindrical portion, said second diameter being larger than said first diameter.

5. A device according to claim 1, wherein the holder is tubular in shape in the first form, whilst at least one end is funnel-shaped in the expanded form.

6. A device according to claim 1, wherein the function element and the holder can be fitted together by a bayonet connection.

7. A device according to claim 1, wherein the holder, in the expanded form thereof, is provided with claws extending away from a central axis.

8. A device according to claim 1, wherein the function element comprises a valve system, by means of which a passage extending through the tubular holder can be opened and closed, respectively.

9. A device according to claim 1, wherein the valve system comprises a valve which is movable in a direction away from a valve seat, which valve system further comprises a damper disposed on a side of the valve remote from the valve seat, said valve being movable in a direction away from the valve seat against the damping force of the damper.

10. A device according to claim 9, wherein the damper comprises a piston which is movable within a cylinder, a piston rod connected to the piston and a valve stop connected to the piston rod on a side remote from the piston, which cylinder has a larger diameter on a side remote from the valve than on a side facing the valve.

11. A device according to claim 10, wherein a liquid is present in said cylinder.

12. A device according to claim 10, wherein the cylinder is a provided with a cap in an end facing the valve, which cap can be moved into the cylinder.

13. A device according to claim 10, wherein the cylinder is a provided with a cap in an end facing the valve, in which cap at least one passage is present, with the damper between said cap and the valve seat being provided with a flexible bellows extending around the piston rod.

14. A device according to claim 1, wherein the function element comprises a respiratory assistance device.

15. A device according to claim 8, wherein the valve system is self-cleaning, with the valve being movable within a valve housing, which valve housing comprises a concave portion near the valve as well as a convex portion adjacent to said concave portion, which convex portion is located on a side remote from the valve seat.

16. A holder suitable for a device according to claim 1.
